(19) United States
(12) Patent Application Publication
(10) Pub. No.: US 2012/0059335 A1
(43) Pub. Date: Mar. 8, 2012

(54) Flexible Anti-Collapsible Catheter Sleeve

(75) Inventor: Donald E. Bobo Sr., Fountain Valley, CA (US)

(73) Assignee: INNERSPACE MEDICAL ,INC. a corporation

(21) Appl. No.: 13/148,944

(22) PCT Filed: Feb. 10, 2010
(86) PCT No.: PCT/US2010/023777

§ 371 Date: Nov. 14, 2011

Related U.S. Application Data
(63) Continuation of application No. 12/431,631, filed on Apr. 28, 2009, now abandoned.
(60) Provisional application No. 61/151,415, filed on Feb. 10, 2009.
(60) Provisional application No. 61/151,415, filed on Feb. 10, 2009.

(51) Int. Cl. A61M 25/00

(52) U.S. Cl. 604/264

(57) ABSTRACT

A thin-walled, spiral-cut sleeve is placed on a portion of a ventricular catheter that may be moved into the compression fitting (or similar securing mechanism) of a bolt in a patient. The wall of the sleeve is sufficiently thick so as to prevent the compression fitting from collapsing the drainage lumen of the catheter. A spiral cut in the sleeve allows the sleeve to flex axially, reducing torque forces on the bolt.

### RELATED APPLICATIONS

This application claims priority to International patent Application No. PCT/US2010/023777, International Filing Date 10 Feb. 2010, entitled Flexible Anti-Collapsible Catheter Sleeve, which claims priority to U.S. patent application Ser. No. 12/431,631 filed Apr. 28, 2009 entitled Flexible Anti-Collapsible Catheter Sleeve (now abandoned), which claims priority to U.S. Provisional Application Ser. No. 61/151,415 filed Feb. 10, 2009 entitled Flexible Anti-Collapse Catheter Sleeve, all of which are hereby incorporated herein by reference in their entireties.

### BACKGROUND OF THE INVENTION

Ventricular catheters are typically used for monitoring pressure and draining fluid (e.g., cerebrospinal fluid) in mammalian bodies, an example of which is seen in U.S. Pat. No. 6,673,022, the contents of which are incorporated by reference. Hence, these catheters typically have one or more passages within them to allow for drainage, air communication, wires or other components.

The ventricular catheter for intracranial use is typically fixed in place with a bolt and compression fitting. One end of the bolt is screwed into or otherwise fixed in the skull of a patient and the other end of the bolt connects to the compression fitting. Once the catheter has been placed in the brain, the compression fitting is tightened to fix the location of the catheter relative to the bolt to prevent axial movement of the catheter within the patient. Since the compression fitting applies pressure to a portion of the catheter, an exoskeleton or similar rigid support structure must be placed over any portion of the catheter that may be contacted by the compression element. This exoskeleton or rigid support structure prevents the catheter's lumen (e.g., such as a drainage lumen) from collapsing.

Previous exoskeleton designs have employed a rigid sleeve or support tube fixed over that portion of the catheter that may be subjected to the force of the compression fitting. This rigid sleeve prevents collapse of the catheter lumens but also remains relatively unbendable. In this respect, the rigid tube maintains the orientation of the catheter in line with the axis of the passage through the bolt and compression fittings. Since the length that the catheter that must be advanced into the brain varies from person to person, the rigid tube must be long enough to accommodate these various catheter positions. Hence, the rigid tube causes at least a portion of the catheter to rigidly stick up from the bolt and compression fitting.

In some arrangements of this system, the bolt, fitting and catheter can rigidly extend away from the patient for some distance. For example, the bolt and fitting may extend above the scalp about 1.5 inches while the rigid tube extends above the bolt by another 1 inch.

This combined length of the bolt, the fitting and the rigid tube is problematic for at least two reasons. First, it increases the likelihood that the assembly will be inadvertently hit. For example, the catheter provides a longer and more rigid area for a nurse or agitated patient to contact.

Second, the length of the tube increases the length of the lever arm which conveys torque to the skull. In this respect, the force of contact from the rigid tube is much greater than it would be against the bolt alone. In some cases, the torque increase allows even a relatively minor force to pop out the bolt from the skull, causing serious complications.

In addition to torque forces, the increased area of the rigid tube may increase the likelihood of applying downward, axial force on the catheter. This force may overcome the holding force of the compression fitting, pushing the catheter into the patient's brain and likely causing damage.

### SUMMARY OF THE INVENTION

According to a preferred embodiment of the present invention, an exoskeleton consisting of a thin-walled spiral-cut sleeve or series of rings is placed on a portion of a ventricular catheter that may be moved into the compression fitting (or similar securing mechanism) of a bolt in a patient. The exoskeleton prevents the compression fitting from collapsing the lumens (e.g., the drainage lumen) of the catheter. Spacing between the rigid areas (such as a spiral cut) allows the exoskeleton to flex axially so that the catheter can bend freely. The flexibility of the exoskeleton lowers the profile of the system and precludes the possibility that a downward force on the catheter will push the catheter into the brain.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 illustrates a catheter and exoskeleton according to the present invention;

FIG. 2 illustrates the catheter and exoskeleton of FIG. 1 with a bolt and compression fitting;

FIG. 3 illustrates a magnified cross sectional view of the compression fitting from FIG. 2;

FIG. 4 illustrates the catheter, exoskeleton, bolt and compression fitting of FIG. 2;

FIG. 5 illustrates the exoskeleton of FIG. 1;

FIG. 6 illustrates the exoskeleton of FIG. 5 in a bent configuration; and

FIG. 7 illustrates an exoskeleton according to the present invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

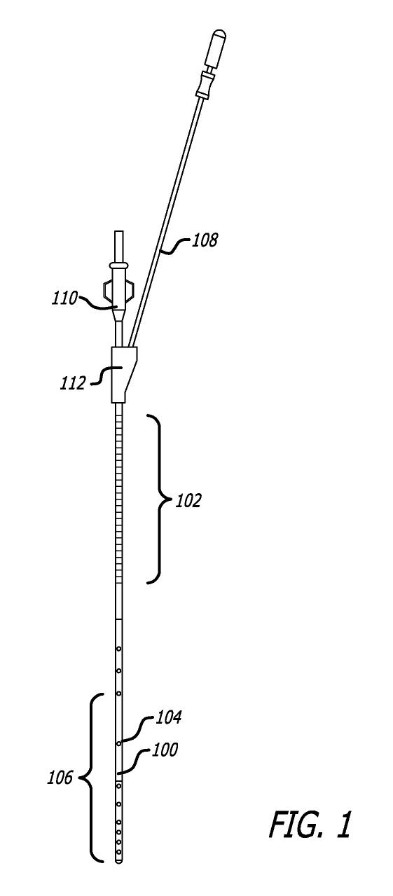

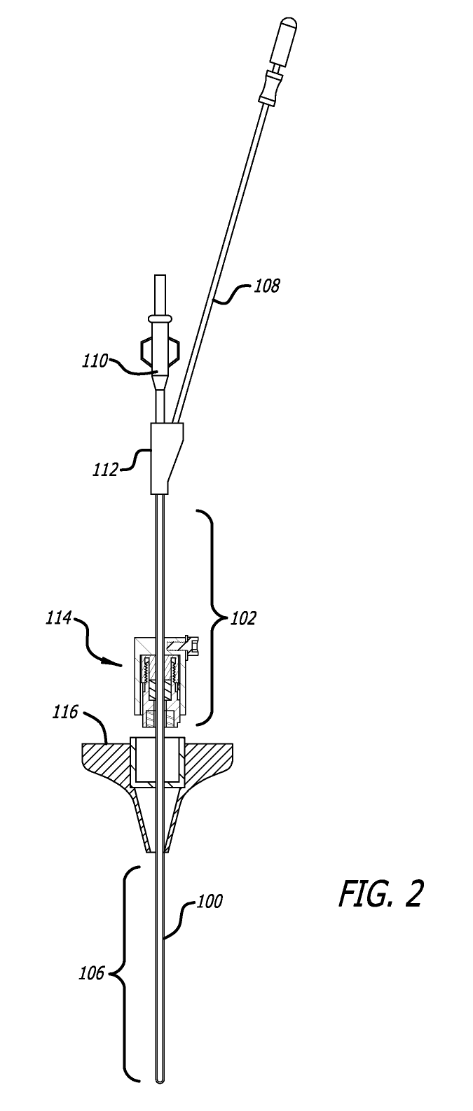

FIGS. 1 and 2 illustrates a catheter 100 having a flexible exoskeleton 102 that allows the catheter 100 to freely bend while preventing the interior contents of the catheter 100 from being crushed by a fastening mechanism such as a compression fitting 114. The exoskeleton 102 reduces the height and therefore the torque that forces (such as accidental contact) can exert on the bolt 116. Hence, the risk of popping out the bolt 116 from, for example, the patient's skull, is greatly reduced.

Preferably, the exoskeleton 102 can be an integral part of the catheter 100 by, for example, adhesive bonding.

As seen in the present example, a distal section 106 of the catheter 100 includes a plurality of drainage apertures which connect to a drainage passage within the catheter 100. The catheter 100 preferably includes a pressure sensor 104 for measuring a pressure within a patient. A tube 108 within which the pressure signal is conveyed, splits off from the catheter 100 at splitter 112. The proximal end of the catheter is terminated in a luer fitting 110. The luer fitting is connected to a standard drainage bag system (not shown).

As best seen in FIG. 2, the exoskeleton 102 is located along a length of the catheter 100 where a compression fitting 114 or similar position fixing mechanism may press against or otherwise compress the catheter 100. Since different patients and different insertion locations may require the catheter 100 to be inserted to different depths, the flexible region extends along much of the length of the catheter 100. Preferably, this exoskeleton 102 length is about 4 inches.

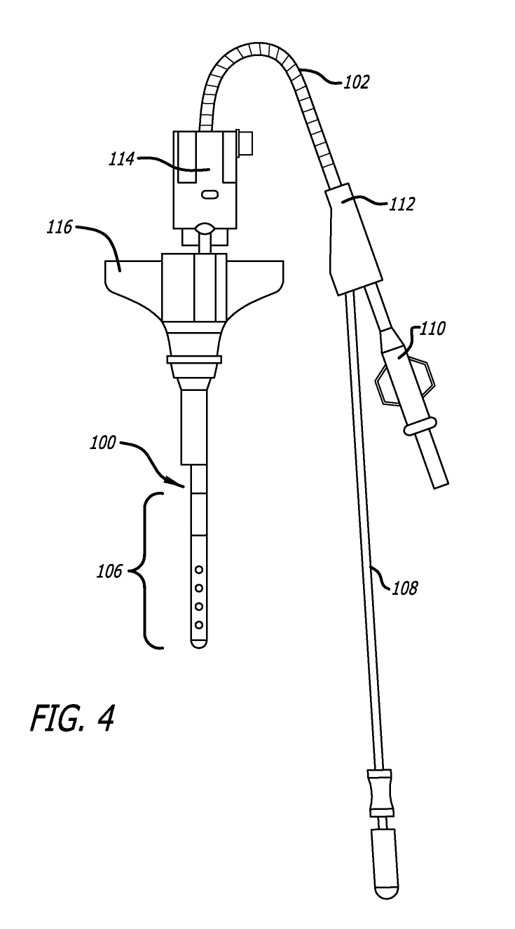

FIG. 4 illustrates the exoskeleton 102 and catheter 100 in a bent or flexed position proximal to the bolt 116 and compression fitting 114. As compared with the non-flexed position in FIG. 2, the exoskeleton 102 reduces torque-amplified forces on the bolt 116 and compression fitting 114 (coupled to the bolt 116) that would otherwise be present if the exoskeleton 102 was non-flexible (as in the prior art).

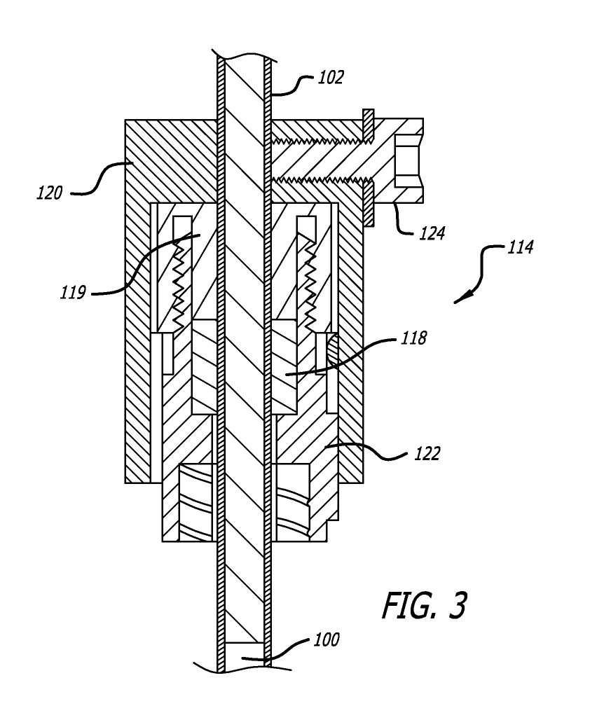

FIG. 3 illustrates a magnified view of a typical compression fitting 114. As the upper portion 120 is screwed onto the lower portion 122, an inner member 119 presses down on a compression element 118. As the compression element 118 is compressed or squeezed downwards, it expands outward against the exoskeleton 102 of the catheter 100. Additionally, a set screw 124 can be further used to further secure the exoskeleton 102 from axial movement.

As previously discussed, prior art exoskeletons are rigid, especially along the length that is squeezed or pressed on by the compression fitting 114. This leaves the prior art exoskeletons unable to bend. However, the exoskeleton 102 resists crushing while allowing flexibility (i.e., axial flexibility along a length of said exoskeleton 102) by preferably includes a plurality of rigid sections or areas that are interspersed with non-rigid areas or even no material. These rigid sections can be connected together as a unitary rigid element or can be distinct from each other. The rigid sections are arranged along the length of the exoskeleton 102 to withstand being crushed by a radial force typically generated by a compression fitting 114. Spaces between the rigid sections allow the exoskeleton 102 to flex as needed.

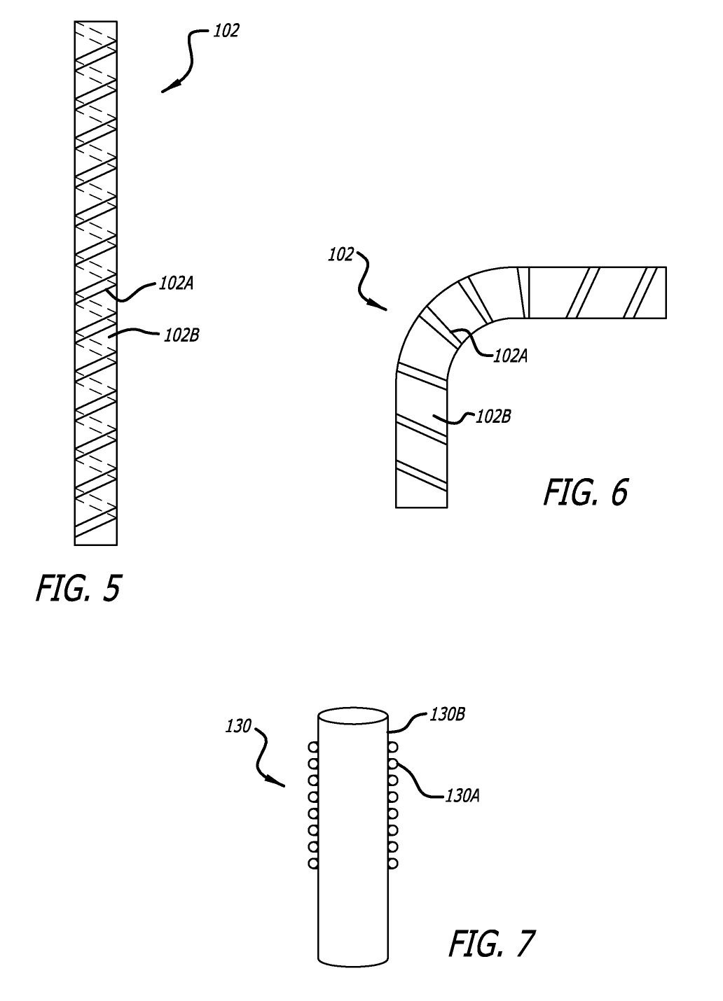

FIGS. 5 and 6 best show a preferred embodiment of the flexible exoskeleton 102, including a spiral cut 102A forming a generally larger spiral of rigid material 102B. This spiral cut 102A introduces axial flexibility into the exoskeleton 102 while retaining much of the strength along the diameter to resist crushing under pressure from the compression fitting 114.

The width of the rigid material 102B can be varied to increase or decrease the crush resistance and flexibility of the exoskeleton 102. Generally, the flexibility can be increased and the crush resistance can be decreased by increasing the number of turns in the spiral cut 102A. Conversely, the crush resistance can be increased and the flexibility can be decreased by decreasing the number of turns in the spiral cut 102A. Preferably, the flexible section 102 sized to fit an 8 French catheter is composed of a rigid material such as polyimide with a thickness of about 0.006″. The spiral cut 102A is preferably about 0.01″ wide and forms about 10 turns per inch.

Preferably, the exoskeleton 102 can be formed by cutting the spiral cut 102A into the tube via a laser or mechanical cutting device. Alternately, this spiral shape can be preformed by molding techniques.

While a spiral cut 102A has been described, it should be understood that other cut shapes are contemplated within the present invention. For example, right angle cuts forming a stair pattern, a spiral wave pattern, a circumferential wave pattern, or similar variations on these patterns.

FIG. 7 illustrates another preferred embodiment of an exoskeleton 130 that includes a plurality of rigid rings 130 (some of which are shown cross sectioned in this figure) which are fixed in place by a flexible tube 130B. The rings 130 are preferably composed of a rigid polyimide or metal and are preferably adhered or embedded within the flexible tube 130B. The flexible tube 130B is preferably composed of a flexible plastic. Alternately, the rings 130 may be only connected by a plurality of longitudinal wires connected to the inner or outer diameter of the rings 130A or may simply be adhered to the exterior of the catheter 100 in a evenly spaced arrangement.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A catheter system comprising:
a bolt at least partially disposed within a patient and having a bolt passage therethrough;
a compression fitting coupled to said bolt and having a fitting passage therethrough and connected to said bolt passage;
a catheter disposed through said bolt and said compression fitting;
a catheter exoskeleton having an inner diameter and being disposed along a length of said catheter at a position contactable by said compression fitting; said catheter exoskeleton having a plurality of rigid portions spaced apart from each other;
wherein said compression fitting is compressed against said catheter exoskeleton; wherein said catheter exoskeleton maintains said inner diameter; and wherein said catheter exoskeleton flexes along a longitudinal axis of said catheter exoskeleton.

2. The catheter system of claim 1, wherein said catheter exoskeleton comprises a tubular shape having a spiral cut.

3. The catheter system of claim 1, wherein said catheter exoskeleton comprises a plurality of rings dispose on a flexible tube.

4. The catheter system of claim 2, wherein said catheter exoskeleton is composed of a rigid polyimide.

5. The catheter system of claim 2, wherein said catheter exoskeleton comprises a polyimide material having a wall thickness of about 0.006 inches.

6. The catheter system of claim 2, wherein said spiral cut forms about 10 turns per inch.

7. The catheter system of claim 6, wherein said spiral cut is about 0.01 inches wide.

8. A catheter system comprising:
a bolt member having a proximal end for fixing within a patient and a distal end having a compression fitting;
a catheter framework disposed through said bolt member and compression fitting, said catheter framework comprising a rigid material and a cut in said rigid material so as to allow a portion of said rigid material to axially flex without being crushed by pressure from said compression fitting;
a catheter located within said catheter frame work.

9. The catheter of claim 8, wherein said cut in said rigid material further comprises a helical cut.

10. The catheter of claim 9, wherein said helical cut forms about 10 turns per inch.

11. The catheter of claim 10, wherein said rigid material is polyimide having a thickness of about 0.006 inches.

12. The catheter of claim 10, wherein said cut is about 0.01 inches wide.

13. A catheter system comprising:
a bolt at least having a distal end configured for anchoring within a skull of a patient and a bolt passage located through said bolt;
a compression fitting disposed on a proximal end of said bolt and having a mechanism for compressing against the contents of a fitting passage, said fitting passage in communication with said bolt passage;
a catheter disposed through said bolt passage and said fitting passage; and,
an exoskeleton comprising a plurality of rigid sections and said exoskeleton being disposed over a length of said catheter contactable by said compression fitting.

14. The catheter system of claim 13, wherein said plurality of rigid sections are formed by a helical gap.

15. The catheter system of claim 13, wherein said plurality of rigid sections comprise a plurality of rigid ring members spaced apart from each other.

16. The catheter system of claim 13, wherein said plurality of rigid sections are unitary with each other.

17. The catheter system of claim 13, wherein said exoskeleton further comprises a helical cut forming a plurality of rigid, curved, helical sections.
